(19) United States
(12) Patent Application Publication
(10) Pub. No.: US 2007/0275383 A1
(43) Pub. Date: Nov. 29, 2007

(54) Novel Hybrid Probes with Heightened Luminescence

(76) Inventors: Francis Vocanson, Aurec Sur Loire (FR); Roger Lamartine, Villeurbanne (FR); Pierre Jean Debouttiere, Massieux (FR); Christophe Marquette, Lyon (FR); Loic Blum, Caluire (FR); Stephane Roux, Pont De Cheruy (FR); Olivier Tillement, Fontaines Saint Martin (FR); Pascal Perriat, Lyon (FR)

Correspondence Address: CLARK & BRODY, 1090 VERMONT AVENUE, NW, SUITE 250, WASHINGTON, DC, 20005, US

(21) Appl. No.: 10/581,052

(22) PCT Filed: Nov. 26, 2004
(86) PCT No.: PCT/FR04/03039

§ 371 Date: Mar. 30, 2007

(30) Foreign Application Priority Data
Nov. 28, 2003 (FR) 0313978

(51) Int. Cl. C12Q 1/68; C12Q 1/00; G01N 33/53

(52) U.S. Cl. 435/6; 435/4; 435/7.2; 435/7.6; 977/774

(57) ABSTRACT

Hybrid probe particles comprising a nanoparticle of gold of diameter in the range extending from 2 to 30 nm on the surface of which, on the one hand, at least one, and preferably from one to 100, organic probe molecules are grafted by gold-sulphur bonds and on the other hand, at least 10, and preferably 10 to 10000, molecules with luminescent activity, as well as their preparation process.

The present invention relates to the technical field of probes for the detection, followup and quantification in biological systems. More particularly, the object of the invention is novel hybrid probe particles whereof the core is constituted by a nanoparticle of gold on which probe molecules are immobilised on the one hand and on the other hand molecules with luminescent activity, as well as their preparation process.

The use of probes associated with a marker, in biological systems for detection (recognition) or followup of specific substances, known as targets, is a common technique in the field of medical diagnostics and research into biology. Such probes are utilised particularly for flux cytometry, histology, immunological tests or fluorescent microscopy, as well as for studying biological materials and non-biological materials.

Common marking systems are for example radioactive iodine isotopes, phosphorous and other elements such as peroxidase enzyme or alkaline phosphatase whereof the detection requires a particular substrate. In the majority of cases, selective coupling between the marker and the substance to be detected is undertaken by a single or an association of functional molecules. The selectivity of the bond is essential to identify without ambiguity the substance target to be detected. The reactions ensuring coupling are known and described for example in <<Bioconjugate Techniques>>, G. T. Hermanson, Academic Press, 1996 or in <<Fluorescent and Luminescent Probes for Biological Activity. A Practical Guide to Technology for Quantitative Real-Time Analysis>>, Second Edition, W. T. Mason, ed., Academic Press, 1999.

Organic fluorescent dyes are widely utilised for marking. These can be fluorescein, Texas Red or Cy5, which are selectively connected to a determined biological or organic substance acting as a probe. After excitation of the probe marked by an external source, most often electromagnetic, the presence of the target biological or organic substances connected to the probe is revealed by the emission of fluorescence on the part of the latter.

The lowering of the detection thresholds constitutes a major objective which would lead to improvement of biochips (analysis and identification of biomolecules) and to the development of more efficient probes capable of ensuring individual tracking of target biomolecules, so as to study their cellular activity, or able to reveal the interactions existing between unicellular beings (bacteria, protozoa . . . ) and minerals which manifest via local physico-chemical modifications of the environment (variation in pH, ionic force, oxygen concentration).

The current limitation on the lowering of detection thresholds is the difficulty in functionalising a biomolecule or a particular site of a biological substrate, constituting the target to be detected, by more than a fluorescent organic function (most often a molecule).

To lower the detection threshold, it is proposed in the prior art to mark the probe intended to be connected to the target to be detected, with intrinsically luminescent particles. In particular, nanoparticles of semi-conductor material have given rise to intense research. U.S. Pat. No. 5,990,479, and the international patent applications published under the numbers WO 00/17642 and WO 00/29617 show that fluorescent semiconductor nanocrystals, which belong to the class of elements II-VI or III-V and those which, under certain conditions, are composed from elements of the 4th principal group of the periodic table, can be utilised as fluorescent marker for biological systems. Due to the phenomenon known as <<quantum size effect>> the emission wavelength of a fluorescent semiconductor nanocrystal is imposed by its size. Therefore, by varying the size of these nanocrystals, a large range of the spectrum can be covered of the visible light close to infrared. Their utilisation as biological marker is described by Warren C. W. Chan, Shuming Nie, Science, 281, 2016-2018, 1998, and by Marcel Bruchez Jr, Mario Moronne, Peter Gin, Shimon Weiss, A. Paul Alivisatos, Science, 281, 2013-2016, 1998. The preparation of semiconductor nanocrystals with a well-defined emission wavelength, that is, with a low-size dispersion, demands a high degree of precision and requires perfect mastery of the operating conditions and the development of synthesis. They are, consequently, very difficult to produce. The extended palette of colours offered by semiconductor crystals results from a variation in size of the order of a few Angström (that is a few atomic layers). The syntheses in solution rarely reach such a degree of precision. In addition, the recombination of electron-hole pairs observed at the surface of the nanocrystals limits the quantic yield at a low value.

To avoid this problem, a core/shell structure has been proposed: it seeks to individually encase the fluorescent semiconductor nanocrystals in a layer of semi-conductor material with a wider gap (ZnS, CdS). In addition, selective marking of biomolecules by fluorescent semi-conductor nanocrystals requires the formation of a layer of polysiloxane functionalised by amine groups (epoxy and carboxylic acid). The latter will constitute anchoring points for the biomolecules. The preparation of these nanocrystals requires, therefore, at least three steps of synthesis whereof the first two are very delicate, and is therefore difficult to commercialise.

Marking by oxide nanoparticles rendered luminescent due to doping by luminescent ions (rare earth) is not widespread yet, despite prospective results. Its main drawback is the low quantic yield which requires the use of a laser to excite the luminescent ions present in the crystalline matrix. On the other hand, the properties of luminescence are very clearly altered, when these particles are utilised directly in an aqueous medium.

Marking by vesicles or balls polymer, or polysiloxane, filled with luminescent organic compounds is efficacious for luminescence visualisation, but often requires fairly large particles (several tens of nanometres) and is delicate to use in certain applications where greater <<molecularity>> is preferred.

Different strategies using grafted particles of gold have already been developed. However, none of these has succeeded in satisfactorily increasing the luminescence emitted. The majority of works has been focused on marking and detection of oligonucleotides whereof one of the ends has been modified by a thiol function. If the grafting of an oligonucleotide strand constitutes a common step in the different strategies specified in the prior art, the means employed for detection are highly variable.

In fact, Pileni et al. in J. Phys. Chem B, 107, 27, 6497-6499, 2003 describe the immobilisation of nanoparticles functionalised by oligonucleotide strands thiolated by hybridisation with the complementary strand present on nanometric islets of gold deposited on a glass surface. The immobilisation (and consequently detection of the oligonucleotide) is revealed by a significant increase in the sensitivity of resonance transmission spectroscopy of the surface plasmon (T-SPR). The electrochemical detection of oligonucleotides was likewise considered by Li et al. in Analyst, 128, 917-923, 2003 and Hsing et al. in Langmuir 19, 4338-4343, 2003. The immobilisation of nanoparticles of gold functionalised by oligonucleotide strands on biochips (by hybridisation) facilitates the germination of silver crystals (by reduction of the argent cation salts (I)) causing an increase in the detection current.

The optical properties of gold have likewise been made good use of for marking and detection. Therefore, Richards-Kortum et al. in Cancer Research, 63, 1999-2004, 2003, showed that nanoparticles of gold could be utilised for detection cancerous cells. In fact, the immobilisation on the nanoparticles of biomolecules interacting selectively with cancerous cells produces probes whereof the detection is based on the capacity of the nanoparticles to reflect incident light emitted by confocal microscope. The nanoparticles of gold can be utilised as an optical contrast agent due to the optical absorption and reflection properties associated with plasmons of gold. Another approach has been developed by Mirkin et al. in J. Am. Chem. Soc. 125, 1643-1654, 2003 who demonstrated that hybridisation of two complementary oligonucleotide strands, carried by two distinct particles of gold, caused connection of these particles and therefore displacement of the plasmon band (resulting from the collective oscillations of the electrons of the conduction band). The change in colour of the colloid (from red to violet) can be made good use of for detection of oligonucleotides in solution or on DNA biochips.

Dubertret et al. in Nature Biotechnology, 19, 365-370. 2001, based their work on the extinction of fluorescence observed for certain organic dyes adsorbed on gold for preparing DNA probes. They showed that hybridisation of an oligonucleotide strand marked by a fluorophore and immobilised on the surface of the gold with a free strand helped to restore the luminescence of the fluorophore, due to the latter moving away from the surface of the gold, generated by hybridisation. The emission of a light of wavelength characteristic of organic fluorophore indicates the presence of the free oligonucleotide. This technique by luminescence extinction helps detect the presence of oligonucleotide in solution.

The encasing of the metallic core by a layer of polysiloxane type was likewise undertaken in WO 99/01 766. However, the process employed does not overcome the homogeneity of the polysiloxane layer making the controlling of the surface of the nanoparticle and therefore the controlling of the number of molecules which could be grafted thereon more difficult.

All these approaches of the prior art are restrictive, since they can be applied in certain conditions only. Electrochemical detection does not allow the a biomolecule becoming in vivo. To be followed. The technique of Mirkin et al. is limited to the detection of nucleic acids. Also, the displacement of the plasmon band can be caused by other factors (increase of the concentration in salt, temperature, ageing).

In this context, one of the problems proposed for resolving the invention is to provide novel biological probes of nanometric size enabling detection, marking and quantification, in vitro and in vivo, in biological systems, with sensitivity and reproducibility.

Another problem, proposed to resolve the invention, is to provide novel biological probes which are easily detectable, due to their fluorescence emission or luminescence exacerbated after excitation.

The invention likewise attempts to provide novel polyfunctional biological probes of controlled size and composition, produced according to a simple process, easily commercialised.

To achieve these objectives, the invention proposes novel hybrid probe particles comprising a nanoparticle of gold having a diameter in the range extending from 2 to 30 nm, on the surface of which, at least one, and preferably 1 to 100, organic probe molecules are grafted by gold-sulphur bonds on the one hand and on the other hand, at least 10, and preferably 10 to 10000 organic molecules with luminescent activity.

The invention likewise proposes a novel type of probe where the exacerbated luminescence is coupled to a dense nanometric metallic core, allowing another investigation system such as the electronic transmission microscopy and/or bases on the properties of reflection, absorption and/or diffusion associated with plasmons.

The object of the invention is likewise different processes fore preparation of hybrid probe particles such as defined hereinabove.

The following description, with reference to the attached figures, will better aid understanding of the object of the invention.

FIG. 1 shows the persistence of luminescence of derivatives of lissamine rhodamine B after grafting on nanoparticles of gold.

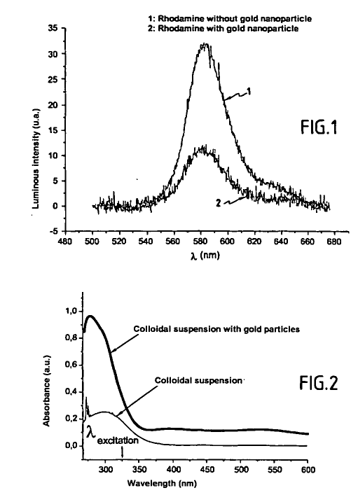

FIG. 2 shows the absorption spectra of a colloidal solution of nanoparticles of gadolinium oxide separate or associated with nanoparticles of gold.

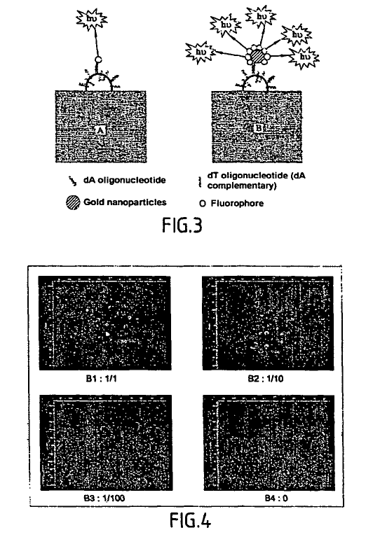

FIG. 3 is a schematic illustration of the principle of the biochip utilised.

FIG. 4 shows the influence of dilution (Laser Argon, λ_{exc}=480 nm, P=600 μW) during immobilisation by hybridisation on a biochip of nanoparticles of gold functionalised by 5 molecules with luminescent activity (thiolated derivative of lissamine rhodamine B) and by an oligonucleotide.

FIG. 5 shows fluorescence observed after immobilisation on Sepharose balls by hybridisation of nanoparticles of gold comprising an oligonucleotide and a variable number of molecules with luminescent activity (thiolated lissamine rhodamine B: rhoda-SH).

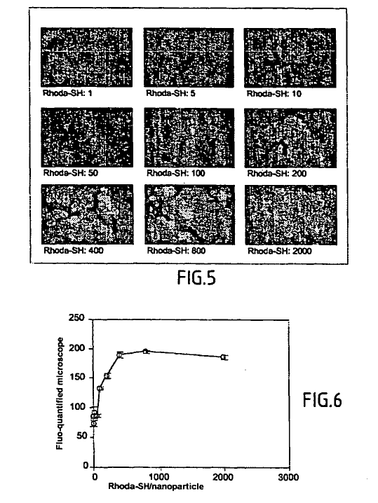

FIG. 6 shows the quantification of the fluorescence signal observed in FIG. 5.

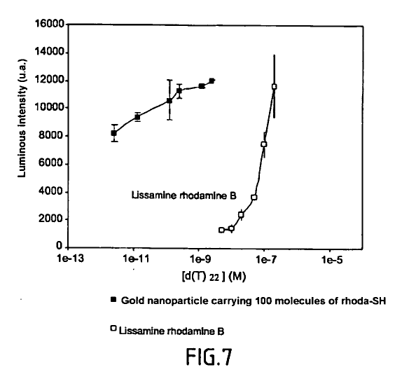

FIG. 7 compares the luminous intensity obtained after marking oligonucleotide by a single molecule with luminescent activity (derivative of lissamine rhodamine B) and by a nanoparticle of gold comprising 100 molecules with luminescent activity (thiolated lissamine rhodamine B).

As a preliminary, the definitions of certain terms used in the present patent application are given hereinbelow.

The terms <<molecule with activity luminescent>>, <<fluorophore>>, <<dye>>, <<fluorescent molecule>> will be utilised variously to designate entities which are possible to detect due to their optical emission activity in the visible and the near infrared.

<<Organic>> molecule is understood as the classic definition well known to the specialist, namely a carbonated molecule optionally containing one or more elements selected from among: O, N, P, S and halogen. The compounds based on silicon and/or metals are naturally not part of the organic molecules.

Probe molecule is understood as a compound which has at least one recognition site allowing it to react with a target molecule of biological interest.

The term “polynucleotide” signifies chaining of at least 2 desoxyribonucleotides or ribonucleotides optionally comprising at least one modified nucleotide, for example at least one nucleotide comprising a modified base, such as inosine, methyl-5-desoxycytidine, dimethylamino-5-desoxyuridine, desoxyuridine, diamino-2,6-purine, bromo-5-desoxyuridine or any other modified base enabling hybridisation. This polynucleotide can also be modified at the internucleotidic bond, the skeleton. Each of these modifications can be taken in combination. The polynucleotide can be an oligonucleotide, a natural nucleic acid or its fragment such as DNA, ribosomic RNA, messenger RNA, transfer RNA, a nucleic acid obtained by an enzymatic amplification technique.

“Polypeptide” is understood to mean chaining of at least two amino acids.

The term “protein” includes holoproteins and heteroproteins such as nucleoproteins, lipoproteins, phosphoproteins, metalloproteins and glycoproteins both fibrous and globular, enzymes, receptors, enzyme/substrate complexes, glycoproteins, antibodies, antigens.

The term “antibody” includes polyclonal or monoclonal antibodies, antibodies obtained by genetic recombination and fragments of antibodies.

The term “antigen” designates a compound likely to be recognised by an antibody from which it has caused synthesis by an immune response.

Nanoparticle is understood to mean a particle of nanometric size. These nanoparticles can be of any form. The particles of spherical formed are, nevertheless, preferred.

The core of the hybrid probe particles according to the invention is constituted by a nanoparticle of gold, preferably of average diameter in the range extending from 2 to 30 nm, preferably in the range extending from 4 to 20 nm and preferably in the range extending from 5 to 16 nm. Since the average size is deduced here by photon correlation spectroscopy (quasi-elastic diffusion of light, λ=633 nm) and by analysis of masters undertaken by electronic transmission microscopy (ETM). The utilisation of gold is particularly advantageous for the following reasons:
gold is compatible with living organisms and has a fairly high tolerance threshold,
gold is a metal very difficult to oxidise, producing nanoparticles having considerable stability (especially the conservation of its state of zero oxidation and metallic behaviour),
synthesis of nanoparticles of gold is easy,
gold is non-paramagnetic,
gold has a particular affinity for sulphur, making grafting of thiolated derivatives possible, the gold-sulphur bond being known to be particularly strong,
gold is visible in ETM imagery,
gold has surface plasmon absorption, producing information on the nanoparticle, especially on its size.

These nanoparticles of gold are polyfunctionalised by grafting of different thiolated derivatives which contribute:
biological recognition given by grafting of at least one, preferably one to 100 and preferably 1 to 10, organic probe molecules,
luminescence in biological medium given by grafting of at least 10, preferably 10 to 10000, preferably 10 to 1000, organic molecules with luminescent activity, advantageously 100 to 500,
solubility adapted as a function of the work medium,
redispersion,
non-aggregation.

Functionalising is easy, the different grafted molecules being connected quasi-covalently to the nanoparticle of gold by gold-sulphur bonds. Within the scope of the invention, the different molecules (probe molecules, molecules with luminescent activity, or other organic molecules) are connected, either directly to the nanoparticle by a Au—S bond, or by means of an organic molecule acting as a spacer, connected to the nanoparticle for a Au—S bond.

If, currently, the nature of the Au—S bond remains undetermined, it is all the same recognised that the thiolate groups are strongly connected to the surface of the gold. According to Dubois, and Nuzzo in Ann. Phys. Chem. 43, 437-, 1992 and Ulman A. in Chemical Reviews 96, 1533-1554, 1996, the bond energy is 40 kcal.mol⁻¹ (as against 87 kcal.mol⁻¹ for the S—H bond) and the energetic balance of the adsorption of an alkanethiolate on gold is negative (˜−5 kcal.mol⁻¹, exothermic reaction). The Au—S interaction created after grafting of thiolated derivatives is so strong that the latter cannot be expelled from the surface by successive washings. The utilisation of thiolated derivatives therefore appears particularly appropriate to immobilise molecules of dyes and biomolecules at the surface of nanoparticles of gold.

A large number of organic molecules with luminescent activity is grafted on surface of nanoparticles of gold. By way of advantage, the number of molecules with luminescent activity grafted on surface of the nanoparticle of gold is at least 10 times greater than the number of grafted organic probe molecules.

In addition, in terms of the invention, the organic molecules with luminescent activity, likewise called dyes, are fixed on the gold either directly (in this case the dyes are thiolated) or indirectly by means of a short organic spacer (the spacer preferably being a thiolated molecule comprising between 2 and 50 carbon atoms). The dyes are therefore not bonded to an oligonucleotide or to a DNA fragment, as described in the international application published under the number WO 03/027678. In accordance with the invention, the dyes are bonded quasi-covalently on the nanoparticle of gold by gold/sulphur bond. By this method, the fluorescence of the dyes is preserved after grafting and is not reduced by the presence of gold which absorbs sharply to 520 nm, not the case of compounds previously selected and adsorbed directly onto the gold. In addition, the luminescent function is ensured by a large number of organic molecules with luminescent activity grafted on the nanoparticle of gold, resulting in a strong fluorescence emission after excitation, producing final global luminescence per widely heightened object. The hybrid nanoparticles according to the invention thus become visualisable at one and the same time in confocal microscopy due to absorption or reflectivity (optical contrast agent) and in electronic microscopy (electronic contrast agent).

In fact, first, the target biomolecule is more easily located because, instead of being marked by a single fluorophore, it is “marked” by several tens of luminescent molecules. A biochip composed of Sepharose balls carrying oligonucleotide (d(A)₂₂), immobilised on the surface of an elastomer (FIG. 3) is utilised to disclose the amplification obtained due to the utilisation of nanohybrid probe particles according to the invention carriers of derivatives of lissamine rhodamine B and oligonucleotides. The strands complementary to those immobilised on the surface of the biochip are marked, either by a single fluorophore molecule (lissamine rhodamine B) (FIG. 3A), or by a hybrid nanoparticle according to the invention carrying a multitude (2-200) of thiolated molecules of lissamine rhodamine B (FIG. 3B and FIG. 4). FIGS. 5 and 6 clearly show the increase in fluorescence with the number of organic fluorescent molecules (lissamine rhodamine B functionalised by a thiol function). However, beyond 400 fluorescent molecules, the intensity ceases to increase and conserves the measured value for nanoparticles of gold on which 400 organic molecules fluorescent are immobilised. These results were obtained on nanoparticles of gold of a 12 nm diameter.

As shown in FIG. 3, following the hybridisation reaction between complementary strands, for the same number of marked strands having reacted with the immobilised strands, that is the same number of target molecules in the sample, an intensity of greater fluorescence is expected. This is illustrated in FIG. 7 presenting the variation in intensity of fluorescence obtained as a function of the quantity of strands present in the sample, marked by either a molecule of lissamine rhodamine B, or by a hybrid probe particle according to the invention. In this very case, several strands can be present on the surface of the nanoparticle, but it is admitted that it will be possible for a single one of these strands to react with an immobilised strand. The curve presented in FIG. 7 takes into account these parameters and supposes that a hundred strands are present on the surface of the nanoparticle, a single one reacting with the immobilised strand. As is evident, an increase in the signal by a factor of ten can be obtained between the molecules marked by a fluorophore (white square) and those marked by hybrid probe particle according to the invention carrying a hundred fluorophores (black square). Despite the partial absorption of the luminous signal emitted by the organic dye due to the colloid of gold, an increase in intensity by a factor of 10 is observed.

In accordance with a first advantageous variant of the invention, the grafted dyes emit on a wavelength located outside the maximum absorption of the plasmon of gold (at 540 nm).

By way of advantage, the molecules with luminescent activity are fluorescent organic dyes whereof the maximum emission deviates by at least 25 nm from the maximum absorption of the gold plasmon. Electroluminescent or chemiluminescent compounds, for example derivatives of luminol, could be utilised. Luminescent compounds, with two photons or with anti-stokes emission, whereof the wavelength of the light emitted is greater than the excitation wavelength, preferably of at least 200 nm, could likewise be grafted. The lanthanide complexes, derivatives of rhodamine and more particularly those of lissamine rhodamine B are particularly preferred dyes.

As evident in FIG. 1, the grafting of lissamine rhodamine B and its derivatives on nanoparticles of gold causes a drop by only a factor of 3 in the intensity of luminescence obtained, as compared to the same quantity of free dyes (individualised molecules). By boosting the number of molecules of grafted lissamine rhodamine B, the luminescence per biological molecule to be detected is further increased.

In accordance with another advantageous variant embodiment of the invention the non-radiative transfers between the organic dyes and gold are limited, so as to obtain nanoparticles with reduced luminescence extinction. For this, at most 75% can be recovered for example of the nanoparticle of gold of a cover material exhibiting dielectric characteristics allowing dislocation of the plasmon band of gold outside the emission zone of the molecules with luminescent activity. This cover material is, for example, selected from among polysiloxanes, SiO₂, ZrO₂, Ln₂O₃ and lanthanide oxohydroxides. The cover must be partial, so as to leave on the nanoparticle of gold a free sufficiently significant surface for the grafting of luminescent and biological molecules. In fact, the organic probe molecules and the luminescent molecules are grafted directly onto the particle of gold and not onto the cover material. FIG. 2 shows, by way of illustration, how grafting by gadolinium oxide can eliminate the absorption of the surface plasmon in the visible field.

Another method of obtaining nanoparticles with reduced luminescence extinction is to graft the molecules with luminescent activity by means of a thiolated organic spacer. The utilisation of organic dyes previously grafted onto <<rigid spacers>> (thiolated organic molecules comprising for example a benzene cycle) keeps the luminescent centre at an average distance of the surface greater than 0.5 nm. These spacers contain, preferably, at least 6 carbons and fewer than 50, and are for example selected from among mercaptophenols, dihydrolipoic acid and thio-poly(ethyleneglycol).

Moreover, the nanohybrid probe particles according to the invention are relatively photostable.

The probes according to the invention are perfectly adapted to a large diversity of biological targeting, the specificities being dependent on the nature of the probe molecules grafted onto the surface of the nanoparticle of gold. The biological probe molecules are advantageously selected from among polynucleotides of type DNA, RNA or oligonucleotides, proteins antibody type, receptor, enzyme, enzyme/substrate complex, glycoproteins, polypeptides, glycolipides, oses, polyosides and vitamins. Oligonucleotides which are thiolated or bonded to a thiolated spacer are particularly preferred. The organic probe molecules can likewise be any type of molecules allowing biotin-streptavidin interaction.

It is likewise possible to graft other organic thiolated molecules, distinct from the organic probe molecules and the molecules with luminescent activity onto the nanoparticle of gold. These organic thiolated molecules preferably comprise at least one alcohol, amine, sulphonate, carboxylic acid or phosphate function. The choice could be made to graft 1 to 1000, preferably, 10 to 1000, of these other organic molecules. The functions contributed by these other molecules are for example better stability, solubility adapted as a function of the work medium, easy redispersion, non-aggregation, better selectivity.

The invention therefore astutely combines nanoparticles of gold, biological probe molecules and molecules with luminescent activity, such that the luminescence is not <<destroyed>> by the absorption of the gold, but on the contrary is overall augmented relative to an isolated molecule (effect of the number of grafted compounds) and the probe molecules retain their efficacy vis-à-vis biological targets.

The hybrid nanoparticles of gold according to the invention are easily synthesised by the Frens method (citrate), of which there are numerous variants (citrate/tannic acid) or by the Brust method known as NaBH₄.

For the citrate method, reference can be made for example to Nature Physical Science 241, 20-22, 1973. In this case, the reduction in hydrogen tetrachloroaurate by citrate in aqueous phase provides nanoparticles of gold covered in citrate. The latter plays a double role: it allows control of the growth in nanoparticles and prevents the formation of aggregates. The citrate/tannic acid association likewise provides nanoparticles covered in citrate whereof the dimensions are smaller. The grafting of thiolated molecules onto the nanoparticles of gold takes place by progressive replacement of the citrate molecules due to portionwise addition of the solution of thiolated molecules. This step is delicate since excessively rapid replacement causes precipitation of the nanoparticles. The immobilisation of different thiolated molecules occurs in as many steps (one step=complete addition of a solution of thiolated species) as there are different molecules.

For the NaBH₄ method, reference could be made especially to J. Chem. Soc., Chem. Commun., 1655-1656, 1995. The NaBH₄ method essentially consists of reacting in an aqueous medium and in the presence of sodium borohydride and hydrogen tetrachloroaurate with the thiolated derivatives to be grafted. The grafted thiolated derivatives are prepared according to methods well known to the specialist. Thiolated derivatives are understood to mean an organic molecule comprising at least one thiol function-SH. These thiol functions can be obtained from dialkyl sulphides or dialkyl disulphides.

These different methods are well known to the specialist, who could add numerous variants to them. In a non-limiting way, a description of different advantageous variants of the process is given hereinbelow.

In accordance with a first variant, the preparation process of hybrid probe particles according to the invention comprises the following steps:
preparing a colloidal suspension of nanoparticles of gold of a diameter in the range extending from 2 to 30 nm, by reduction of a gold salt, and in particular of hydrogen tetrachloroaurate, in an aqueous or alcoholic phase and in the presence of citrate,
adding to the resulting colloidal suspension an aqueous or alcoholic solution of thiolated organic probe molecules grafting onto the surface of the nanoparticles of gold by a gold-sulphur bond replacing citrate molecules,
adding to the resulting colloidal suspension an aqueous or alcoholic solution of molecules with luminescent activity grafting onto the surface of the nanoparticles of gold by a gold-sulphur bond replacing citrate molecules.

In the case of the citrate and citrate/tannic acid methods, preparation of the hybrid probe particles comprises at least three steps. Advantageously, the first consists of preparing in an aqueous phase particles of gold of a nanometric size generally between 10 and 20 nm according to the citrate method and between 6 and 15 nm according to the citrate/tannic acid method, and this, advantageously, by reduction of HAuCl₄.3H₂O by citrate (citrate method) in a Au/Citrate ratio of between 0.170 and 0.255 and by the citrate/tannic acid (citrate/tannic acid method) couple in Au/citrate and tannic acid/citrate ratios of between 0.170 and 0.255 and between 0.030 and 10 respectively. The nanoparticles of gold are then covered by molecules of citrate adsorbed on their surface. The colloids can optionally be purified by dialysis against water.

In the case of the citrate and citrate/tannic acid methods, the functionalising of the nanoparticles is carried out in several steps. Each step corresponds to the grafting of a single sort of molecule. The grafting is undertaken by replacement of the citrate present on the surface of the nanoparticles, and therefore requires gradual addition of the solution containing the molecules to be grafted comprising a thiol function. The quantity of molecules grafted onto the nanoparticles of gold is advantageously between 0.1 and 60% of the free sites.

The grafting of molecules having biological activity, for example thiolated oligonucleotides, folic acid modified by a thiol function or grafted onto thiolated poly(ethylene glycol) (PEG), is preferably carried out by the addition of 1 to 500 μl of an aqueous concentration solution of between 0.1 μM and 40 μM. The grafted quantity of probe molecules on the surface of the nanoparticles of gold is advantageously between 1 and 200 probe molecules per particle.

The second step consists of grafting the organic dye carrying one or more thiol functions, preferably by addition of 3 to 200 μl of an aqueous (or ethanolic) solution of the thiolated concentration dye of between 0.1 and 400 μM. The number of grafted thiolated dyes is advantageously between 10 and 400 per particle, for particles of a diameter of 12 nm especially.

The grafting of biological probes can equally be effected before or after that of the dyes. The solutions of different thiolated species such as sodium mercaptoethanesulphonate, succinic acid, PEG terminated by a thiol function can optionally be added successively before, between or after the two preceding steps and in any order. When functionalising is complete, the hybrid nanoparticles of gold are purified by column chromatography (Sephadex™ G-25 M, eluent: buffer solution of pH of between 7 and 9).

In accordance with another variant of the citrate or citrate/tannic acid method, the process comprises the following steps:
preparing a colloidal suspension of nanoparticles of gold of a diameter in the range extending from 2 to 30 nm, by reduction of hydrogen tetrachloroaurate, in an aqueous or alcoholic phase and in the presence of citrate,
adding to the resulting colloidal suspension an aqueous or alcoholic solution of thiolated spacers functionalised with an ionisable function likely to react with the organic probe molecules or the molecules with luminescent activity to be grafted, said spacers grafting onto the surface of the nanoparticles of gold by a gold-sulphur bond replacing citrate molecules,
adding an aqueous or alcoholic solution of organic probe molecules functionalised to react with the ionisable function carried by the spacers grafted onto the surface of the nanoparticle of gold,
and/or adding an aqueous or alcoholic solution of organic probe molecules functionalised to react with the ionisable function carried by the spacers grafted onto the surface of the nanoparticle of gold.

Instead of grafting a thiolated organic molecule having luminescent or biological activity directly onto the surface of a nanoparticle of gold, this other variant consists of carrying out the grafting by condensation between two complementary reactive functions present for one in the active molecule to be grafted (dye, probe . . . ) and for the other at the end of a thiolated molecule immobilised on the surface of the gold and acting as a spacer. The grafting of an organic molecule with luminescent or biological activity requires the presence of a thiol function to ensure its lasting immobilisation on the gold particle. The majority of these molecules are deprived thereof. The thiol function can be introduced by organic synthesis before grafting (case of the citrate protocol). Another way to proceed consists of grafting the active molecule deprived of thiol function onto a thiolated spacer present at the surface of the nanoparticle of gold. Relative to the preceding protocol the one step of grafting active thiolated molecules is replaced by two steps. The first consists of immobilising the thiolated spacer acting as anchor point (spacer arm) on the molecule having luminescent or biological activity. Advantageously, from 1 to 500 μl aqueous solution of the concentration spacer of between 0.1 and 400 μM is then added to the colloid of nanoparticles of gold. The number of immobilised thiolated molecules is advantageously between 0.1% and 50% of free sites.

Next, an aqueous solution of the active molecule to be grafted is added slowly. This solution can optionally contain a reagent facilitating coupling. The elimination of secondary products is done by dialysis of the colloidal solution against water. The spacer utilised as grafting site must necessarily comprise a thiol function (indispensable for immobilisation on gold) and at least one reactive function (—OH, —NH₂, —COCl . . . ) to ensure subsequent grafting of the active molecule. To obtain the best luminescence results, the carbonated chain between the thiol function and the reactive function must be rigid and preferably comprises from 6 to 50 carbon atoms. The organic molecule having luminescent or biological activity must necessarily comprise a reactive function (—SO₂Cl, —COCl, —OH, —NH₂) capable of reacting with that carried by the spacer arm immobilised on the surface of the nanoparticles of gold. Reference can especially be made to Chem. Eur. J, 8, 16, 3808-3814, 2002 and Chem. Commun. 1913-1914, 2000.

Irrespective of the protocol utilised (active thiolated molecule or thiolated spacer), the number of molecules with luminescent activity immobilised on the surface of the nanoparticles is determined by UV-visible spectroscopy of the solution after precipitation of the nanoparticles. The difference between the number of molecules added to the colloid and the number of molecules present in the surfactant (after filtration of the precipitate) indicates the number of molecules immobilised on the surface of the nanoparticles of gold.

In accordance with another variant utilising the NaBH₄ method the process comprises the following steps:
preparing a colloidal suspension of nanoparticles of gold of a diameter in the range extending from 2 to 30 nm, by reduction of gold salt, and in particular of hydrogen tetrachloroaurate, in an aqueous or alcoholic phase and in the presence of NaBH₄,
adding to the resulting colloidal suspension an aqueous or alcoholic solution of spacers thiolated functionalised with an ionisable function likely to react with the organic probe molecules or the molecules with luminescent activity to be grafted, said spacers grafting onto the surface of the nanoparticles of gold by a gold-sulphur bond,
adding an aqueous or alcoholic solution of organic probe molecules functionalised to react with the ionisable function carried by the spacers grafted onto the surface of the nanoparticle of gold,
adding an aqueous or alcoholic solution of organic probe molecules functionalised to react with the ionisable function carried by the spacers grafted onto the surface of the nanoparticle of gold.

In the case of the NaBH₄ method the thiolated molecules present on the surface of the nanoparticles of gold have in general been introduced during synthesis. Certain of these can be substituted, though with uncertain control of the number of molecules replaced. The immobilisation of molecules with biological activity and organic dyes will take place in the majority of cases (for improved efficacy) by grafting on thiolated spacers present on the surface of the nanoparticles of gold. Synthesis by the NaBH₄ method of hybrid nanoparticles for biological marking likewise requires several steps. The first consists of preparing, methanol, ethanol or dimethylformamide preferably in an alcohol, with the nanoparticles of gold covered in thiolated molecules having a function ionisable in a single step by reduction of HAuCl₄.3H₂O by an aqueous solution of NaBH₄ (Au/NaBH₄, for example between 0.05 and 0.5) in the presence of organic thiolated molecules having an ionisable function whereof the Au/S ratio is, advantageously, between 0.2 and 10. With this method, covering the nanoparticles of gold is quasi-total.

As replacement of the thiolated molecules on the surface of the nanoparticles of gold is difficult and as the surface is completely covered, the choice of thiolated molecules is decisive. These molecules must allow both excellent redispersion of the nanoparticles in an aqueous solution to obtain a stable colloid, and grafting of probe molecules and organic dyes. Thiolated spacers likewise have an ionisable function (—NH₂, —COOH) and appear appropriate for preparing hybrid nanoparticles of gold which are redispersible and stable (under certain pH conditions) in an aqueous solution. In addition, these ionisable functions can act to immobilise probe molecules and organic dyes by simple condensation reactions (formation of ester, amide, derivatives of urea or thiourea . . . ).

After reduction and therefore formation of the nanoparticles of gold, a precipitate results. At a maximum ⅔ of the solvent (methanol or ethanol) are then evaporated under reduced pressure at a temperature of less than 40° C. The precipitate is filtered on a polymer membrane (with, for example, a diameter of pores equal to 0.22 μm) and washed meticulously with different solvents (selected according to the nature of the thiol immobilised on the surface). This washing aims to eliminate the co-of the reduction and the large quantity of non-adsorbed thiols.

The powder obtained is, after air drying, redispersed in an aqueous phase in a controlled pH range (which depends on the nature of the ionisable group present in the thiolated molecule). The organic dye is then grafted onto the nanoparticle by reaction between a reactive function, of the type —NH₂, —COOH, —SO₂Cl, —N═C═O, —N═C═S especially, present on the dye and the ionisable function of the thiolated spacer grafted onto the nanoparticles of gold. This reaction is made by adding to the colloidal solution an aqueous solution or aquo-alcoholic of organic dye whereof the quantity is at least four times greater than the number of thiolated molecules adsorbed on the nanoparticles of gold. Between 0.5 and 10% of the ionisable functions of the thiolated molecules adsorbed on the gold react in general. The secondary products in excess are then eliminated by precipitation of the nanoparticles obtained by a strong variation in the pH (ΔpH≧2). The precipitate is filtered on a membrane (diameter of the pores is equal to 0.22 μm for example) and washed thoroughly prior to being redispersed in an aqueous solution in a controlled pH range.

The probe molecules are grafted onto a part of 85 to 90% of the ionisable functions remaining after grafting of the organic dye. The coupling is made by addition of an aqueous solution of probe molecules whereof the quantity is at least greater than the number of thiolated molecules adsorbed on the nanoparticles of gold. Between 0.1 and 2% of the ionisable functions of the thiolated molecules grafted onto the gold react. To avoid denaturation by the repetition of the separation, washing and redispersion steps, grafting of the probe molecules is advantageously carried out after grafting of the organic dyes. The secondary products in excess are eliminated as previously.

Characterisation of the nanoparticles is undertaken in the solid state by XPS, XANES, ATG and in the liquid state by UV-visible spectroscopy and XANES.

Another variant of the process consists of immobilising the probe molecules by exchange of probe molecules thiolated with other thiolated molecules already grafted on the surface of the nanoparticles of gold. To avoid harmful exchange with part of the thiolated molecules coupled to dyes, it is indispensable in this case to undertake immobilisation of the molecules having biological activity prior to that of the organic dyes. However, these exchange reactions are relatively random and difficult to manage.

It should be noted that in the NaBH₄ method covering the nanoparticles of gold by the thiolated molecules is quasi complete. The introduction of novel thiolated molecules is consequently done purely by exchange.

It is important to note that for the nanohybrid probe particles prepared in the presence of citrate there is no significant exchange of thiols: the nanohybrids are therefore stable in these cases and the properties are retained. The proposed method therefore determines the <<coating>> of the nanoparticle of gold, and therefore the characteristics of the resulting particle probe.

In accordance with the invention, it is possible to graft onto the surface of the nanoparticles of gold variable but determined quantities of fluorescent molecules and biological probes. The number of molecules at the surface of the nanoparticles can be easily determined by UV spectroscopy after precipitation of the particles of gold, thus allowing the chemical composition of the surface to be known.

The novel probe particles according to the invention have a quite particular interest, especially in the improvement of biochips, the study of the interaction between microorganisms and their environment, the individual tracking of biomolecules for the study of cellular traffic and cellular activity.

The examples hereinbelow are given purely by way of illustration and are not limiting in nature.

### EXAMPLE 1

### Preparation of a Colloidal Suspension of Nanoparticles of Gold by the Citrate/Tannic Acid Method

40 mg of sodium citrate and 10 mg tannic acid are dissolved in 20 ml of ultra-pure water. In parallel, 10 mg hydrogen tetrachloroaurate, trihydrate HAuCl₄.3H₂O are dissolved in 80 ml of ultra-pure water. The two solutions are then heated at 60° C. then combined by racking of the sodium citrate/tannic acid solution in the gold solution. The mixture is then heated at 60° C. at reflux for 1 hour, then brought to the boil for 10 minutes and finally cooled to room temperature under continuous stirring. The nanoparticles obtained have an average diameter of 8 nm, which reflects a concentration of 1.67.10⁻⁸ moles of nanoparticles/litre.

### EXAMPLE 2

### Preparation of Nanoparticles of Gold Stabilised and Ready to be Functionalised by Grafting of Thiolated Derivatives

The surface of the synthesised nanoparticles according to Example 1 is covered by thiolated derivatives in precise proportions. The thiolated derivatives utilised are sodium mercaptoethanesulphonate (MES), thiomaleic acid (AT) and mercaptophenol (MP). 2 ml of aqueous solutions of each thiolated derivative are added to a solution of 60 ml of nanoparticles whereof the concentrations are the following:
AT: 1.112.10⁻⁷ M obtained by dissolution of 16.69 mg in 100 ml of deionised water,
MES: 1.112.10⁻⁷ M obtained by dissolution of 18.26 mg in 100 ml of deionised water,
MP: 2.224.10⁻⁷ M obtained by dissolution of 28.50 mg in 100 ml of deionised water.

The additions are made successively every 30 minutes, and the solution is kept under constant agitation.

### EXAMPLE 3

### Preparation of a Colloidal Suspension of Luminescent Nanoparticles of Gold by the Citrate/Tannic Acid Method in the Same Conditions as Those of Example 2

Fluorescent molecules of rhodamine lissamine B are immobilised on the hydroxyl functions of the mercaptophenols grafted onto the surface of the nanoparticles of gold. 1 ml of an aqueous solution of lissamine rhodamine B thiochloride having a concentration of 10⁻⁷ M in the presence of 10 ml of concentrated triethylamine is added to 30 ml of solution prepared according to Example 2. The result is nanoparticles of gold carrying on average 200 molecules of lissamine rhodamine B.

### EXAMPLE 4

### Synthesis of a Thiolated Derivative of Rhodamine Lissamine B.

This derivative is obtained by reaction of the amine function of aminothiophenol on the thiochloride function of rhodamine lissamine B. The reaction occurs at room temperature by dissolution of 125 mg lissamine rhodamine B thiochloride and 26.9 mg aminothiophenol in 100 ml chloroform in the presence of 1 ml triethylamine. The solution is agitated for a day, then purified by silicon column chromatography with dichloromethane/methanol eluent, 9/1 (v/v).

### EXAMPLE 5

### Grafting of Thiolated Derivatives of Lissamine Rhodamine B Prepared According to Example 4 on the Surface of Nanoparticles of Gold Prepared According to Example 1

The preparation is undertaken by addition of thiolated solutions of lissamine rhodamine B to the solution of nanoparticles of gold with mechanical agitation. This addition is variable in quantity and concentration according to the number of desired fluorescent molecules per nanoparticle; this number can vary from 1 to 400 for nanoparticles of 12 nm in diameter. For example, for a desired ratio of 100 molecules of lissamine rhodamine B per nanoparticle, addition will be 1 ml of an aqueous solution to 1.67.10⁻⁵ M thiolated lissamine rhodamine B on 10 ml of a solution at 1.67.10⁻⁸ M of nanoparticles of gold.

### EXAMPLE 6

### Grafting of a Derivative of Folic Acid with Sulphide Termination

A sulphur derivative of folic acid is obtained by grafting bis-aminopropylpolyethyleneglycol, then modification by the Traut reagent to obtain a thiol function. This derivative is grafted to the surface of nanoparticles of gold by addition to a solution of nanoparticles prepared according to Example 1.

### EXAMPLE 7

### Grafting of Oligonucleotides on Nanoparticles of Gold

The utilised oligonucleotides d(T)22 terminated by a thiol function are previously filtered on column, and 69 nanomoles oligonucleotides diluted in 2.33 ml water, or a concentration of 29.6.10^{<6} M, are recovered. From 3.35 μl to 335.1 μl (from 0.2 to 20 oligonucleotides per nanoparticle) of this solution are then added to 1 ml of nanoparticles of gold prepared according to Example 1, 3 or 5.

### EXAMPLE 8

### Grafting of Thiolated Derivatives of Lissamine Rhodamine B Prepared According to Example 4 on the Surface of Nanoparticles of Gold Prepared According to Example 7

Derivatives of thiolated lissamine rhodamine B are grafted onto the nanoparticles prepared according to Example 7, prepared according to Example 4 in a ratio of 100 for one nanoparticle of gold. This grafting is completed as described previously in Example 5.

### EXAMPLE 9

### Synthesis of Particles of Gold Partially Surrounded by Particles of Gadolinium Oxide for Shifting the Absorption Outside the Emission Zone of Molecules with Grafted Luminescent Activity

The colloid of nanoparticles of Gd₂O₃ 5% Tb³⁺ has been prepared according to the polyol (method R. Bazzi, M. A. Flores-Gonzalez, C. Louis, K. Lebbou, C. Dujardin, A. Brenier, W. Zhang, O. Tillement, E. Bernstein and P. Perriat in Journal of Luminescence 102-103, 445-450. 2003). It consists of directly precipitating nanoparticles of luminescent oxides from metallic salts dissolved in diethylene glycol. After synthesis, the colloid obtained is dialysed at 40° C. in diethylene glycol (1:20 in volume).

Next, HAuCl₄, 3H₂O is dissolved in the colloid (1:3 in mass of initial salts). The solution is agitated for 15 minutes, to turn it yellow. Two aqueous solutions first containing 1 g.l⁻¹ sodium citrate and 1.5 g.l⁻¹ tannic acid and secondly 0.5 g.l⁻¹ NaBH₄ are prepared to reduce the gold salt.

The first solution is added to the colloid, during agitation. After five minutes, the second solution is added (1:1:1 in volume). The addition is done slowly, dropwise. Throughout the different additions the colloid loses its yellow colour to pass through a transparent phase, then through an intense red phase, which appears progressively, direct proof of the presence of nanoparticles of gold. Under certain conditions, the luminescence can be greatly exacerbated (by at least a factor of 10).

### EXAMPLE 10

### Synthesis of Nanoparticles of Gold Stabilised by Thiolated Molecules Carrying at their End a Carboxylic Acid Function

38 ml methanol containing from 49 to 196.10⁻⁵ mol carboxylic acid carrying one or two thiol functions and 1.96 ml ethanoic acid are added to 60 ml methanol containing 49.10⁻⁵ mol tetrachloroauric acid (HAuCl₄, 3H₂O). After 5 minutes of agitation, 13.2 ml of an aqueous solution containing 480.10⁻⁵ mol sodium tetrahydruroborate (NaBH₄) are added dropwise to the mixture which turns black.

After 1 hour of stirring, 4 ml of an aqueous solution of hydrochloric acid (HCl, 1 N) are added to the reaction mixture. The black suspension obtained is concentrated by partial evaporation of methanol under reduced pressure. The black solid is filtered, washed by 3×30 ml HCl 0.1 N, 2×20 ml water and 3×30 ml diethyl ether. The reaction mixture is then dried at room temperature. The powder obtained can be easily redispersed in an aqueous solution having a pH greater than or equal to 7.

### EXAMPLE 11

### Grafting of Luminol onto Nanoparticles of Gold Prepared According to Example 10

8 mg of nanoparticles of gold (of average diameter equal to 5 nm) are dispersed in 10 ml of an aqueous solution of pH 8-10. 1 ml of a solution of 0.1 M 1-ethyl-3-(3-dimethylamino)propyl carbodiimide (EDC) and 0.2 M pentafluorophenol in propane-2-ol is added to the colloidal solution of nanoparticles of gold. After 90 minutes, 154 μl to 1.54 ml of an aqueous solution are added to 10⁻² M luminol. After 150 minutes, the nanoparticles are precipitated by addition of an aqueous solution of HCl 1 N. The resulting precipitate is filtered and washed before being redispersed in an aqueous solution of pH≧7.

Variant: in place of a solution of propane-2-ol containing 0.1 M EDC and 0.2 M pentafluorophenol, an aqueous solution of 0.1 M EDC and 0.2 M N-hydroxysuccinimide can be utilised.

### EXAMPLE 12

### Grafting of a Thiolated Oligonucleotide on the Nanoparticles Prepared According to Example 11

1.11.10⁻⁹ mol thiolated oligonucleotides are added to 1 ml of a colloidal solution of nanoparticles of gold (6,7.10¹⁷ nanoparticles/litre). After 1 h, the particles are precipitated by addition of nanoparticles of HCl 1 N. The resulting precipitate is filtered and washed before being redispersed in an aqueous solution of pH≧7.

### EXAMPLE 13

### Grafting of an Oligonucleotide Terminated by an Amine Function onto the Nanoparticles Prepared According to Example 11

1 ml of a solution at 0.1 M EDC and 0.2 M pentafluorophenol in propane-2-ol is added to 1 ml of a colloidal solution of nanoparticles of gold (6,7.10⁻⁷ nanoparticles/litre). After 90 minutes, 1.11.10⁻⁹ mol thiolated oligonucleotides d(T)22 terminated by an amine function are added. After 2 and a half hours, the nanoparticles are precipitated by addition of an aqueous solution of HCl 1 N. The resulting precipitate is filtered and washed before being redispersed in an aqueous solution of pH 8-10.

Variant: in place of a solution of propane-2-ol containing 0.1 M EDC and 0.2 M pentafluorophenol, an aqueous solution of 0.1 M EDC and 0.2 M N-hydroxysuccinimide can be used.

1. Hybrid probe particles comprising a nanoparticle of gold of a diameter in the range extending from 2 to 30 nm onto the surface of which are grafted by gold-sulphur bonds, at least one, and preferably from one to 100, organic probe molecules on the one hand, and on the other hand, at least 10, and preferably 10 to 10000, molecules with luminescent activity.

2. The hybrid probe particles as claimed in claim 1, characterised in that the number of molecules with luminescent activity grafted on the surface of the nanoparticle of gold is at least 10 times greater than the number of grafted organic probe molecules.

3. The hybrid probe particles as claimed in claim 1, characterised in that 10 to 1000, preferably 100 to 500, molecules with luminescent activity are grafted onto the nanoparticle of gold.

4. The hybrid probe particles as claimed in any one of claims 1, characterised in that the molecules with luminescent activity are fluorescent organic dyes whereof the emission maximum deviates by at least 25 nm from the absorption maximum of the gold plasmon.

5. The hybrid probe particles as claimed in claim 1, characterised in that the molecules with luminescent activity are electroluminescent or chemiluminescent compounds, for example derivatives of luminol.

6. The hybrid probe particles as claimed in claim 1, characterised in that the molecules with luminescent activity are luminescent compounds whereof the wavelength of the light emitted is greater than the excitation wavelength, preferably at least 200 nm.

7. The hybrid probe particles as claimed in claim 1, characterised in that the molecules with luminescent activity are lanthanide complexes.

8. The hybrid probe particles as claimed in claim 1, characterised in that the molecules with luminescent activity are selected from among the derivatives of rhodamine and in particular those of lissamine rhodamine B.

9. The hybrid probe particles as claimed in claim 1, characterised in that at most 75% of the nanoparticle of gold is covered by a cover material exhibiting dielectric characteristics allowing shift of the plasmon band of gold en outside the emission zone of the molecules with luminescent activity.

10. The hybrid probe particles as claimed in claim 9, characterised in that the cover material is selected from among polysiloxanes, SiO₂, ZrO₂, Ln₂O₃ and lanthanide oxohydroxides.

11. The hybrid probe particles as claimed in claim 1, characterised in that the molecules with luminescent activity are grafted to the nanoparticles of gold by means of a thiolated organic spacer, this spacer not being identical to the organic probe molecules.

12. The hybrid probe particles as claimed in claim 11, characterised in that the spacer contains from 6 to 50 carbon atoms and is for example selected from among mercaptophenols, dihydrolipoic acid and thio-poly(ethyleneglycol).

13. The hybrid probe particles as claimed in claim 1, characterised in that the nanoparticle of gold has a diameter in the range extending from 4 to 20 nm, preferably in the range extending from 5 to 16 nm.

14. The hybrid probe particles as claimed in claim 1, characterised in that 1 to 10 organic probe molecules are grafted onto the nanoparticle of gold.

15. The hybrid probe particles as claimed in claim 1, characterised in that the organic probe molecules are selected from among polynucleotides type DNA, RNA or oligonucleotides, proteins of antibody type, receptor, enzyme, enzyme/substrate complex, glycoproteins, polypeptides, glycolipides, oses, polyosides and vitamins.

16. The hybrid probe particles as claimed in claim 14, characterised in that the organic probe molecules are thiolated oligonucleotides or attached to a thiolated spacer.

17. The hybrid probe particles as claimed in claim 1, characterised in that the organic probe molecules are molecules allowing biotin-streptavidin interaction.

18. The hybrid probe particles as claimed in claim 1, characterised in that 10 to 1000 other organic thiolated molecules, distinct from the organic probe molecules and the molecules with luminescent activity, are additionally grafted onto the nanoparticle of gold, these other organic thiolated molecules preferably comprising at least one alcohol, amine, sulphonate, carboxylic acid or phosphate function.

19. A preparation process for hybrid probe particles as claimed in claim 1, characterised in that it comprises the following steps:
preparing a colloidal suspension of nanoparticles of gold of a diameter in the range extending from 2 to 30 nm, by reduction of a gold salt, and in particular hydrogen tetrachloroaurate, in an aqueous phase or alcoholic and in the presence of citrate,
adding to the resulting colloidal suspension an aqueous or alcoholic solution of thiolated organic probe molecules grafting onto the surface of the nanoparticles of gold by a gold-sulphur bond replacing citrate molecules,
adding to the resulting colloidal suspension an aqueous or alcoholic solution of molecules with luminescent activity grafting onto the surface of the nanoparticles of gold by a gold-sulphur bond replacing citrate molecules.

20. A preparation process for hybrid probe particles characterised in that it comprises the following steps:
preparing a colloidal suspension of nanoparticles of gold of a diameter in the range extending from 2 to 30 nm, by reduction of hydrogen tetrachloroaurate, in an aqueous or alcoholic phase and in the presence of citrate,
adding to the resulting colloidal suspension an aqueous or alcoholic solution of thiolated spacers functionalised with an ionisable function likely to react with the organic probe molecules or the molecules having luminescent activity to be grafted, said spacers grafting onto the surface of the nanoparticles of gold by a gold-sulphur bond replacing citrate molecules,
adding an aqueous or alcoholic solution of organic probe molecules functionalised to react with the ionisable function carried by the spacers grafted onto the surface of the nanoparticle of gold,
and/or adding an aqueous or alcoholic solution of organic probe molecules functionalised to react with the ionisable function carried by the spacers grafted onto the surface of the nanoparticle of gold.

21. A process for preparation of hybrid probe particles as claimed in claim 19, characterised in that the reduction of the gold salt is done in the presence of tannic acid.

22. The process for preparation of hybrid probe particles as claimed in claim 1, characterised in that it comprises the following steps:
preparing a colloidal suspension of nanoparticles of gold of a diameter in the range extending from 2 to 30 nm, by reduction of gold salt, and in particular hydrogen tetrachloroaurate, in an aqueous or alcoholic phase and in the presence of NaBH₄,
adding to the resulting colloidal suspension an aqueous or alcoholic solution of thiolated spacers functionalised with an ionisable function likely to react with the organic probe molecules or the molecules having luminescent activity to be grafted, said spacers grafting onto the surface of the nanoparticles of gold by a gold-sulphur bond,
adding an aqueous or alcoholic solution of organic probe molecules functionalised to react with the ionisable function carried by the spacers grafted onto the surface of the nanoparticle of gold,
adding an aqueous or alcoholic solution of organic probe molecules functionalised to react with the ionisable function carried by the spacers grafted onto the surface of the nanoparticle of gold.
